(19) United States
(12) Patent Application Publication
(10) Pub. No.: US 2025/0151991 A1
(43) Pub. Date: May 15, 2025

(54) ARTICULATING MEDICAL DEVICES AND ASSOCIATED METHODS

(71) Applicant: Boston Scientific Scimed, Inc., Maple Grove, MN (US)

(72) Inventors: Jeffrey C. MARRION, Littleton, MA (US); Kian S. LIM, Shrewsbury, MA (US); Thomas M. ZAPPIA, JR., West Boylston, MA (US); Richard C. TAH, Milford, MA (US); Camron HAGEMEYER, Bloomington, IN (US); Jimmy HARRISON, Greencastle, IN (US); Bradley Randall BLACK, Bloomington, IN (US); Patrick N. LEUGUE, Shrewsbury, MA (US); Niraj Prasad RAUNIYAR, Plymouth, MN (US)

(73) Assignee: Boston Scientific Scimed, Inc., Maple Grove, MN (US)

(21) Appl. No.: 18/939,041

(22) Filed: Nov. 6, 2024

Related U.S. Application Data
(60) Provisional application No. 63/597,491, filed on Nov. 9, 2023.

(51) Int. Cl. A61B 1/008; A61B 1/00; A61B 1/012; A61B 1/05; A61B 1/06; A61M 25/01

(52) U.S. Cl. CPC A61B 1/008; A61B 1/012; A61B 1/00066; A61B 1/05; A61B 1/0676; A61M 25/0138

(57) ABSTRACT

Medical devices and uses thereof are described, including a medical device with a handle, an insertion portion, and a distal tip. The distal tip may include a light source, an imaging device, and a core. First and second articulation members may extend longitudinally from the handle through the insertion portion and the distal tip, e.g., in respective first and second channels of the core. The insertion portion may define a first lumen. The core may include a second lumen between the first channel and the second channel, the second lumen being in communication with the first lumen of the insertion portion. Each of the first articulation member and the second articulation member may terminate proximal to a distalmost end of the core.

### CROSS-REFERENCE TO RELATED APPLICATIONS

This application claims the benefit of priority to U.S. Provisional Application No. 63/597,491, filed on Nov. 9, 2023, which is incorporated by reference herein in its entirety.

### TECHNICAL FIELD

This disclosure relates generally to assemblies for medical devices and associated methods. More specifically, aspects of this disclosure pertain to assemblies including components that are positioned at a distal tip of a medical device, such as an endoscope.

### BACKGROUND

In a medical procedure, an operator may insert a medical device, such as an endoscope or other type of scope, into a body lumen of a subject. The operator may navigate the distal tip to a location where the procedure is to be performed. The operator may pass accessory devices (e.g., instruments) through a working channel of the medical device to assist with performing certain diagnostic or therapeutic procedures. The distal tip of the medical device may include elements for providing visualization of the body lumen, such as lighting elements and/or imaging devices. The distal tip of the medical device also may include other features, such as one or more articulation members, an opening of the working channel, one or more openings for air/water and suction, a sensor, and/or an elevator. Many current scopes present limitations on the arrangement of elements and/or types of elements that may be incorporated into the distal tip of the device. It may be desirable to arrange the articulation members within the distal tip to optimize the space within the distal tip and without increasing an outer diameter of the medical device. For example, it may be desirable to provide room for additional components and/or larger components (e.g., additional and/or larger lighting elements, imaging devices, and/or sensors).

### SUMMARY

Each of the aspects disclosed herein may include one or more of the features described in connection with any of the other disclosed aspects.

Aspects of this disclosure relate to, among other things, assemblies for medical devices and associated methods. Aspects of this disclosure pertain to assemblies including components that are positioned at a distal tip of a medical device, such as an endoscope.

For example, the present disclosure includes a medical device comprising a handle, an insertion portion extending distally from the handle, and a distal tip at a distal end of the insertion portion. A first articulation member and a second articulation member may each extend longitudinally from the handle through the insertion portion. The insertion portion may define a first lumen. The distal tip may include a core and optionally may include a light source and/or an imaging device. The core may include a first channel that receives a distal portion of the first articulation member, a second channel that receives a distal portion of the second articulation member. A second lumen may extend through the core between the first channel and the second channel. The second lumen may be in communication with the first lumen of the insertion portion. Each of the first articulation member and the second articulation member may terminate proximal to a distalmost end of the core. The first articulation member may include a first bend distal to a first distal face defining a distal end of the first channel. The second articulation member may include a second bend distal to a second distal face defining a distal end of the second channel.

The medical devices discussed above and elsewhere herein may include any of the following features. The medical device may further comprise a first distal member fixed to the distal portion of the first articulation member. A proximal face of the first distal member may abut the first distal face of the core. The second distal member may be fixed to a distal end of the second articulation member. A proximal face of the second distal member may abut the second distal face of the core. Each of the first distal face and the second distal face of the core may extend along a plane transverse to a longitudinal axis of the core. Each of the first bend and the second bend may be approximately 90 degrees to approximately 180 degrees. The first bend may be in a first direction, and the second bend may be in a second direction, opposite the first direction. The core may further include a first groove on an external surface of the core that receives the distal portion of the first articulation member, and a second groove on an external surface of the core that receives the distal portion of the second articulation member. Each of the light source and the imaging device may be distal to the distal portion of each of the first articulation member and the second articulation member.

In some aspects, the core may further include a first pocket adjacent to the first channel and a second pocket adjacent to the second channel. The first pocket may be in communication with the first lumen of the core and may receive a first distal member fixed to the distal portion of the first articulation member. The second pocket may be in communication with the second lumen of the core and may receive a second distal member fixed to the distal portion of the second articulation member. A proximal face of the first distal member of the first articulation member may abut a distal end of the first pocket. A proximal face of the second distal member of the second articulation member may abut a distal end of the second pocket.

In some aspects, the core may further include a third channel adjacent to the first channel and a fourth channel adjacent to the second channel. The third channel may receive a portion of the first articulation member after the first bend. The fourth channel may receive a portion of the second articulation member after the first bend. A proximal face of the first distal member may abut a first proximal face of the core that defines a proximal end of the first channel, and a proximal face of the second distal member may abut a second proximal face of the core that defines a proximal end of the second channel. Each of the first proximal face and the second proximal face may extend along a plane transverse to a longitudinal axis of the core. The first articulation member may further include a third bend proximal to the first proximal face of the core. The third bend is in a third direction perpendicular to the first bend. The second articulation member may further include a fourth bend proximal to the second proximal face of the core. The fourth bend is in a fourth direction opposite the third direction.

In some aspects, a cross-sectional dimension of a proximal portion of the first channel may be smaller than a cross-sectional dimension of a distal portion of the first channel, and a cross-sectional dimension of a proximal portion of the second channel may be smaller than a cross-sectional dimension of a distal portion of the second channel. The distal tip may further include a cap that receives at least a portion of the core.

The present disclosure also includes a medical device comprising a distal tip that includes a light source, an imaging device, a first articulation member, a second articulation member, and a core. A distal end of the first articulation member may be fixed to a first ferrule. A distal end of the second articulation member may be fixed to a second ferrule. The core may define a lumen extending along a longitudinal axis of the core, a first channel configured to receive a first articulation member, and a second channel configured to receive a second articulation member. The first channel may be parallel to the lumen. At least one of a first distal face or a first proximal face defined by the first channel may be transverse to the lumen. The second channel may be parallel to the lumen. At least one of a second distal face or a second proximal face defined by the second channel may be transverse to the lumen.

Any of the medical devices described above and elsewhere herein may include any of the following features. A proximal end of the first ferrule may abut the first distal face, and a proximal end of the second ferrule may abut the second distal face. the medical device may further comprise a handle, and an insertion portion extending distally from the handle. Each of the first articulation member and the second articulation member may extend from the handle through the insertion portion to the distal tip. The first articulation member may include a first bend distal to the first distal face, and the second articulation member may include a second bend distal to the second distal face.

The present disclosure also includes a medical device comprising a handle, an insertion portion extending distally from the handle to a distal tip and defining a first lumen. A first articulation member rand a second articulation member may each extend from the handle through the insertion portion. The distal tip may include a light source, an imaging device, and a core. The core may include a first channel that may receive a distal portion of the first articulation member and a second channel that may receive a distal portion of the second articulation member. The first channel may define a first distal face, and the second channel may define a second distal face. Each of the light source and the imaging device may be distal to the distal portion of each of the first articulation member and the second articulation member. The first articulation member may include a first bend distal to the first distal face and the second articulation member includes a second bend distal to the second distal face.

Any of the medical devices described above and elsewhere herein may include any of the following features. Each of the first bend and the second bend may be approximately 90 degrees to approximately 180 degrees. The core may further include a third channel and a fourth channel. The third channel may be adjacent to the first channel. The third channel may receive a portion of the first articulation member after the first bend. The fourth channel may be adjacent to the second channel. The fourth channel may receive a portion of the second articulation member after the first bend.

### BRIEF DESCRIPTION OF THE DRAWINGS

The accompanying drawings, which are incorporated in and constitute a part of this application, illustrate examples of this disclosure and together with the description, serve to explain the principles of the disclosure.

FIGS. 1A and 1B depict an exemplary medical device according to aspects of this disclosure.

FIGS. 2A-2D illustrate another exemplary distal portion of a medical device, according to aspects of this disclosure.

FIGS. 3A-3D illustrate another exemplary distal portion of a medical device, according to aspects of this disclosure.

FIGS. 4A and 4B illustrate another distal portion of a medical device, according to aspects of this disclosure.

FIG. 5 illustrates a perspective view of a further distal portion of a medical device, according to aspects of this disclosure.

FIG. 6 illustrates a perspective view of another distal portion of a medical device, according to aspects of this disclosure.

FIGS. 7A and 7B depict a perspective view (FIG. 7A) and a cross-sectional view (FIG. 7B) of features of another exemplary medical device, according to aspects of this disclosure.

FIGS. 8A and 8B illustrate another exemplary distal portion of a medical device, according to aspects of this disclosure.

FIGS. 9A and 9B illustrate another exemplary distal portion of a medical device, according to aspects of this disclosure.

FIGS. 10A-10C illustrate various views of another distal portion of a medical device, according to aspects of this disclosure.

FIG. 11 illustrates another configuration of a distal portion of a medical device, according to aspects of this disclosure.

FIGS. 12A-12D illustrate another distal portion of a medical device, according to aspects of this disclosure.

### DETAILED DESCRIPTION

It may be understood that both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The drawings may include arrows labeled “P” and “D,” indicating proximal and distal directions, respectively. The term “approximately,” or like terms (e.g., “substantially”), includes values +/−10% of a stated value.

Aspects of the present disclosure provide configurations of articulation components to increase the relative size of a working channel, e.g., to facilitate treatment of a subject. A medical device, such as a ureteroscope, may be inserted into a body lumen of a subject (e.g., into a kidney) in order to perform a medical procedure (e.g., a laser lithotripsy procedure). The distal tip may include a distal portion of one or more articulation members, e.g., the articulation member(s) including one or more pull wires. In some aspects, the distal portion of the articulation member(s) may include a distal member. The distal member may include a ferrule, or a crimp, fixed to the distal portion, e.g., fixed to the distal end, of each articulation member. The articulation member(s) may be configured to articulate the distal tip in one or more directions (e.g., moving the distal tip left, right, up, and/or down).

The distal portion, including the distal tip, of the medical device may include one or more assemblies each including various electronic components, such as illumination or lighting elements (e.g., light emitting diodes (LEDs), optical fibers (e.g., plastic optical fibers (POFs), other light guides, or a combination thereof), imaging elements (e.g., cameras, other components having imagers, and/or other optical elements such as lenses), and/or other electronic components (e.g., capacitors, diodes, resistors, etc.). Electronic components of the assemblies herein may include electrical connections, and may also include various elements mounted, for example, or otherwise connected to, the electrical connections.

It may be desirable for the articulation member(s) to occupy a relatively small, or limited, amount of space while retaining quality of performance and manufacturing. For example, with the articulation member(s) occupying a relatively small amount of space, the medical device may be capable of accommodating additional components (e.g., working channels, lumens, electronic components, and/or other components), and/or have additional space within a cross-section of the distal tip. Additionally or alternatively, with the articulation member(s) occupying a relatively small amount of space within the distal tip, the medical device (e.g., a distal portion of the medical device) may include a smaller overall size to facilitate insertion and/or navigation within the body. For example, the diameter or other cross-sectional size of the medical device may be reduced and/or an overall length of the distal tip may be reduced as compared to previous devices. One exemplary manner of conserving space while retaining quality of performance and manufacturing is to position and/or configure the articulation member(s) in a distal tip or end cap of a medical device (e.g., an endoscope or ureteroscope).

Additionally or alternatively, it may be desirable to configure the articulation member(s) to modify or otherwise compensate for proximal forces being applied to each articulation member. For example, when the medical device is articulated, one or more articulation members may be pulled proximally. As such, tensile forces may be applied. When the tensile forces are greater than the coupling forces between respective articulation members and distal members coupled to (e.g., fixed to) distal portions of the articulation members, separation between the two components may occur. Similarly, one or more articulation member(s) may separate from the distal tip and/or one or more components of the distal tip may crack or break when the tensile forces are applied to the articulation member(s). When such separation, cracking, or breaking occurs, performance of the device may be impacted (e.g., the distal tip and/or other portions of the medical device may no longer be able to articulate).

In some examples herein, including those discussed below, the articulation member(s) may be configured with one or more bends to modify or otherwise compensate for (e.g., decrease) the tensile forces being applied. In some aspects, the one or more bends may decrease the likelihood of separation between each respective articulation member and distal member and/or the separation of each articulation member from the distal tip (e.g., separation from the core of the distal tip).

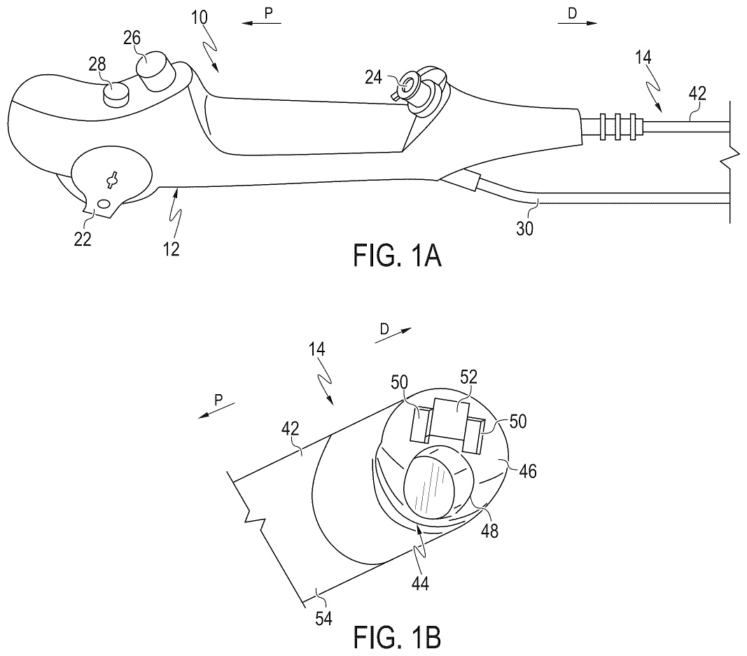

FIGS. 1A and 1B depict aspects of an exemplary medical device 10 according to the disclosure. Although the disclosure may refer at different points to a ureteroscope, it will be appreciated that, unless otherwise specified, endoscopes, duodenoscopes, gastroscopes, endoscopic ultrasonography (“EUS”) scopes, colonoscopes, bronchoscopes, laparoscopes, cystoscopes, aspiration scopes, sheaths, catheters, and any other suitable medical device may include and/or be used in connection with the elements, components, and assemblies described herein. FIG. 1A depicts a proximal portion of medical device 10, including a handle 12 and an insertion portion 14. FIG. 1B depicts a distal portion of insertion portion 14. Handle 12 may be configured for gripping and use by an operator.

Insertion portion 14 may include a shaft 42 extending distally from handle 12. At least a portion of shaft 42 may be flexible, for example, to facilitate navigation through a subject's anatomy, and shaft 42 may define one or more lumens, the lumen(s) optionally containing wires, tubes, or other features passing therethrough. In some aspects, insertion portion 14 may include a single lumen (e.g., a first lumen), through which tubes (e.g., a working channel and/or one or more fluidics tubes), wires, or other features may pass through. Insertion portion 14 may be configured for at least partial insertion into a body (e.g., a body lumen) of the subject.

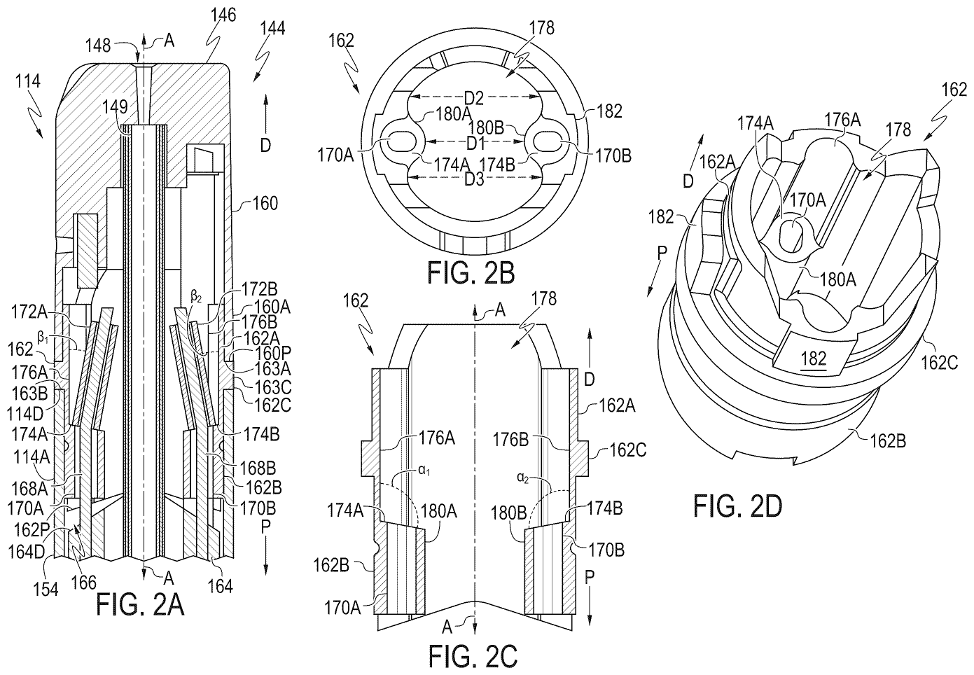

As discussed in further detail below, insertion portion 14 may include one or more electronic components operably connected to one or more components of handle 12. Furthermore, as discussed in detail below, one or more articulation members may extend from handle 12 and through at least a portion of shaft 42 of insertion portion 14. For example, the articulation member(s) may extend longitudinally through insertion portion 14 (e.g., via one or more lumens of shaft 42). In some aspects, the articulation member(s) may be coupled (e.g., directly or indirectly) to a distal tip 44 disposed at a distal end of insertion portion 14. The articulation member(s) may be configured to articulate distal tip 44 in one or more directions. For example, a distal portion of shaft 42 may include an articulation portion 54, which may include one or more articulation joints (see, for example, FIGS. 2A, 3A, 7A, 8A, 9A, and 11), for example, within an outer sheath of shaft 42.

Handle 12 may include a mechanism to control articulation of medical device 10. For example, the mechanism may include an actuator such as lever 22 coupled to the articulation member(s). For example, lever 22 may be coupled to a proximal end and/or a proximal portion of the articulation member(s) such that activation of lever 22 controls articulation/steering of distal tip 44. In some aspects, a proximal portion of handle 12 may include lever 22 as shown in FIG. 1A. It will be appreciated that any suitable actuator(s) may be used in addition to or in place of lever 22, such as one or more knobs, buttons, sliders, switches, or joysticks. Handle 12 may include one or more locking mechanisms, for example, to lock a position of lever 22 and, thus, lock an orientation of articulation portion 54 of shaft 42 (e.g., distal tip 44).

A port 24 of handle 12 may provide access to a lumen (e.g., working channel) of insertion portion 14 of medical device 10. An operator may insert an instrument or other device into port 24 and may extend the instrument or other device distally through the working channel of shaft 42 of insertion portion 14. The working channel may extend longitudinally through a length of shaft 42, for example, and terminate at a distal opening 48 of distal tip 44. In some examples, handle 12 may include a suction valve 26, for example, on a proximal portion of handle 12, e.g., on an opposing side of handle 12 from lever 22. An operator may connect handle 12 to a source of suction, and may operate suction valve 26 to control suction through insertion portion 14 (e.g., through the working channel or other lumen, which may be the same or different from the lumen or working channel in communication with the port 24). Handle 12 may additionally or alternatively include other types of valves, such as, for example, fluid supply valves (e.g., air and/or water valves), and/or valves that perform a combination of functions.

An actuator of handle 12 operably coupled to an imaging device 52 of insertion portion 14 may enable an operator to capture images. For example, as shown in FIG. 1B, handle 12 includes a button 28 to allow the operator to capture still images and/or video images. While button 28 is shown on a proximal portion of handle 12, button 28 (or other actuator) may be on another part of handle 12 accessible to the operator's hand during use.

Still referring to FIG. 1A, an umbilicus 30 may extend from handle 12 and may include or carry wires, cables, and/or conduits configured to provide, for example, power, signals, or fluids to and/or from handle 12. For example, umbilicus 30 may connect handle 12 to one or more user interfaces, monitors, control units, displays, etc.

As shown in FIG. 1B, distal tip 44 may include a distalmost face 46 that defines an opening 48, e.g., a working channel opening. Instruments or other devices may be passed through port 24, through the working channel, and extend out of opening 48. An instrument extending distally of opening 48 may be used to perform a medical procedure on the subject.

Distal tip 44 may also include imaging components, such as one or more light sources 50 (e.g., LED(s)) and one or more imaging devices 52 (e.g., camera(s)). Although two light sources 50 and one imaging device 52 are depicted in FIG. 1B, it will be appreciated that other combinations of light source(s) 50 and imaging device(s) 52 may be utilized. Light sources 50 and imaging device 52 may be separate components or may be combined into a single unit or device. Light sources 50 may include LEDs, optical fibers (e.g., plastic optical fibers (POFs)), or any other suitable light source. Imaging device 52 may be configured to take video and/or still images. Imaging device 52 may provide a signal to a monitor or display (e.g., connected via umbilicus 30), so that an operator may view an image provided by imaging device 52 while navigating medical device 10 through a body lumen of a subject.

As depicted in FIG. 1B, in some aspects, medical device 10 may be forward-facing. For example, features of distal tip 44 (e.g., opening 48, light sources 50, and/or imaging device 52) may face in the same direction, such as, for example, a distal direction (i.e., facing distally/forward of distalmost face 46). This disclosure also encompasses other configurations of distal tip 44. For example, medical device 10 may be side-facing. In a side-facing example, opening 48, light sources 50, and/or imaging device 52 may be disposed on a side of distal tip 44 or otherwise incorporated into distal tip 44 so that they point in a radially outward direction, relative to a longitudinal axis of insertion portion 14. In some aspects, medical device 10 may include one or more components that are forward-facing and one or more components that are side-facing.

FIG. 2A illustrates a cross-sectional view of a distal portion of an insertion portion 114 in an assembled state. Insertion portion 114 may have any or all of the characteristics of insertion portion 14, except as described below. Insertion portion 114 may include a distal tip 144 including a cap 160 and a core 162. In some aspects, core 162 may taper inwards, for example, towards a longitudinal axis A of insertion portion. Cap 160 includes distalmost face 146, which may include one or more light sources and/or one or more imaging devices. A proximal portion 160A of cap 160 may be configured to receive a distal portion 162A of core 162, e.g., via complementary mating features. For example, core 162 may be coupled to cap 160 via mechanical fit (e.g., a snap fit, a press-fit, a screw fit, etc.) and/or with an adhesive. FIGS. 2B-2D illustrate various views of core 162. For example, FIG. 2B illustrates a front, or top-down, view of core 162; FIG. 2C illustrates a side cross-sectional; and FIG. 2D illustrates a perspective view (shown without articulation members 168A, 168B, discussed below). Insertion portion 114 may include an articulation portion 154 proximally of distal tip 144.

Distalmost face 146 may define an opening 148 in communication with a working channel 149, e.g., so as to permit fluid flow from working channel 149 through insertion portion 114. While opening 148 is shown as having a smaller inner diameter than working channel 149, opening 148 may have the same inner diameter or a larger inner diameter as working channel 149. The distal portion of working channel 149 may be tapered, for example, such that opening 148 has a greater diameter than the diameter of working channel 149 proximal to opening 148, or vice versa. In some examples, distalmost face 146 may include additional openings, for example, to permit the delivery of fluids (e.g., gas or liquid) from distal tip 144.

In some examples herein a distal portion 114A of insertion portion 114 may be configured to receive a proximal portion 162B of core 162, e.g., via mechanical fit (e.g., a snap fit, a press-fit, a screw fit, etc.) and/or with an adhesive. In some aspects, a center portion 162C of core 162 may be disposed between insertion portion 114 and cap 160. For example, a proximal face 160P of cap 160 may abut a distal face 163A of center portion 162C. Similarly, a distal face 114D of insertion portion 114 may abut a proximal face 163B of center portion 162C. Center portion 162C may have a greater cross-sectional dimension as compared to distal portion 162A and proximal portion 162B. In some aspects, an outer surface 163C of center portion 162C may form a portion of an outer surface of insertion portion 114.

Articulation portion 154 may include one or more articulation joints 164 disposed within insertion portion 114, for example, proximal of distal tip 144. As such, a proximalmost face 162P of core 162 and a distalmost face 164D of a distalmost articulation joint 164 may be complementarily shaped to one another. For example, proximalmost face 162P may be angled and/or shaped according to distalmost face 164D of a distalmost articulation joint 164. A space 166 may be defined between proximalmost face 165P and distalmost face 164D. Space 166 may change in size (e.g., increase or decrease), for example, when distal tip 144 is articulated.

One or more articulation members may extend through insertion portion 114 and at least a portion of core 162, for example, via one or more channels or lumens. The articulation member(s) may be configured to articulate distal tip 144 in one or more directions. For example, a first articulation member 168A may extend through a first channel 170A of core 162. A second articulation member 168B may extend through a second channel 170B of core 162. A cross-section of first channel 170A and/or second channel 170B of core 162 may be ovular, as shown in FIG. 2B. Alternatively, a cross-section of first channel 170A and/or second channel 170B may be circular, square, rectangular, or any other shape. In some aspects, the cross-section of first channel 170A and second channel 170B may be complementary to a cross-sectional size/shape of each respective articulation member (e.g., first articulation member 168A and/or second articulation member 168B).

In some aspects, each of first articulation member 168A and second articulation member 168B may be loosely contained within first channel 170A and second channel 170B, respectively. For example, each of first articulation member 168A and second articulation member 168B may be movable (e.g., translatable and/or rotatable) within first channel 170A and second channel 170B, respectively. In other aspects, first articulation member 168A and/or second articulation member 168B may be fixed within each respective channel, for example, via an adhesive. Additionally or alternatively, first articulation member 168A and second articulation member 168B may be overmolded or otherwise fixed, within core 162. In some aspects, each of the first articulation member 168A and second articulation member 168B may terminate proximal to a distalmost end of core 162.

Each of first articulation member 168A and second articulation member 168B may include a wire, a cable, a thread, or a cord. In some examples, first articulation member 168A may be coupled to a first ferrule 172A and/or a portion of first articulation member 168A may be crimped. Similarly, second articulation member 168B may be coupled to a second ferrule 172B and/or or a portion of second articulation member 168B may be crimped. Although two articulation members are shown in this example, only one or more than two articulation members may be included, e.g., with correspondence ferrule(s) and/or crimped portions.

Core 162 may be configured to direct at least a distal portion of first articulation member 168A and/or second articulation member 168B inwards, for example, towards longitudinal axis A. For example, a proximal face of first ferrule 172A and/or a proximal face of second ferrule 172B may abut a respective face of core 162, e.g., first angled face 174A and second angled face 174B of core 162. First channel 170A and/or second channel 170B may be centered on first angled face 174A and second angled face 174B, respectively, of core 162.

Each of first angled face 174A and second angled face 174B may be angled inwards within core 162. For example, first angled face 174A may extend along a first plane that is transverse to a longitudinal axis of core 162. Similarly, second angled face 174B may extend along a second plane that is transverse to a longitudinal axis of core 162. For example, an outermost edge of each of first angled face 174A and second angled face 174B (e.g., an edge farthest from longitudinal axis A on each respective surface) may be distal relative to an innermost edge of each of first angled face 174A and second angled face 174B (e.g., an edge closest to longitudinal axis A on each respective surface). In some examples, each of first angled face 174A and second angled face 174B may be angled relative to respective first internal surface 176A and second internal surface 176B of core 162 by angles α₁ and α₂, respectively (e.g., see FIG. 2C). Angles α₁ and α₂ may each greater than 90 degrees to less than 180 degrees, e.g., approximately 91 degrees to approximately 120 degrees. In some aspects, each of first articulation member 168A and second articulation member 168B may be angled relative to first internal surface 176A and second internal surface 176B, respectively, by angles β₁ and β₂, respectively. For example, each of angles β₁ and β₂ may be greater than 0 degrees to less than 90 degrees, e.g., approximately 15 degrees to approximately 45 degrees).

As shown in FIGS. 2A and 2C, first angled face 174A and second angled face 174B may be proximal to a distalmost end of core 162. Accordingly, when a proximal end of first ferrule 172A abuts first angled face 174A and/or when a proximal end of second ferrule 172B abuts second angled face 174B, a distalmost end of first articulation member 168A, second articulation member 168B, first ferrule 172A, and/or second ferrule 172B may be adjacent to or proximal of the distalmost end of core 162. As such, when the distal end of each of first ferrule 172A and second ferrule 172B is adjacent to or proximal of the distal end of core 162, additional space may be created distal to first ferrule 172A and second ferrule 172B within distal tip 144. In some aspects, the additional space may be used to accommodate additional components within distal tip 144 (e.g., imaging devices, light sources, etc.).

A lumen 178 may extend through core 162, for example, from a proximal end of core 162 to a distal end of core 162. Lumen 178 may be centered within core 162, e.g., extending along a central longitudinal axis of core 162. Lumen 178 may be configured to receive and/or contain wires or cables providing electrical connection between light sources 50 and/or imaging device 52 and handle 12. A distal portion of lumen 178 may be circular or ovular. A proximal portion (e.g., the portion of core 162 that includes first channel 170A and second channel 170B) of lumen 178 may resemble a figure eight shape. For example, a first diameter D1 of the proximal portion of lumen 178 may be smaller than a second diameter D2 and/or a third diameter D3 of the proximal portion of lumen 178. First diameter D1 may be smaller, for example, due to a first wall 180A and a second wall 180B formed opposite first wall 180A protruding into lumen 178. First wall 180A may form at least part of first channel 170A. Second wall 180B may form at least part of second channel 170B.

Internal and/or external surfaces of core 162 may include one or more surface features (e.g., protrusions, indentations, grooves, openings, walls, pockets, cut outs, etc.) for example, to assist in coupling cap 160 to core 162 and/or core 162 to insertion portion 114. For example, as shown in FIG. 2D, an outer surface of core 162 may include surface features 182. Cap 160 and/or insertion portion 114 may include complementary features (e.g., mating features), for example, to facilitate with coupling core 162 to insertion portion 114.

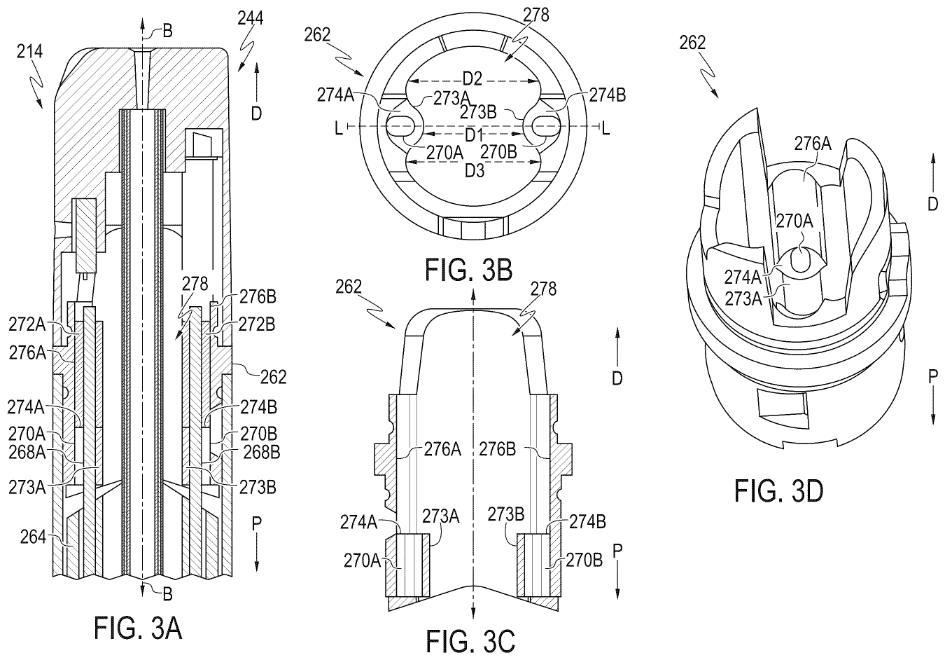

FIG. 3A illustrates a cross-section of another exemplary device according to the present disclosure, showing insertion portion 214. Insertion portion 214 may have any or all of the characteristics of insertion portion 14 and/or insertion portion 114 described above. Insertion portion 214 may include a distal tip 244 coupled to one or more articulation joints 264 of insertion portion 114. Distal tip 244 may include a cap 260 and a core 262, which may have any or all of the characteristics of cap 160 and core 162, respectively, except as described below. For example, in this configuration, core 262 may be configured to align one or more articulation members parallel to a longitudinal axis B. With the one or more articulation members aligned parallel to longitudinal axis B, additional space may be created, for example, distal to the one or more articulation members and/or within a cross-section of distal tip 144.

FIGS. 3B-3D illustrate various views of core 262 including a front, or top-down, view in FIG. 3B; a side cross-sectional view in FIG. 3C; and a perspective view in FIG. 3D (omitting articulation members for clarity). Core 262 may be configured to orient a first articulation member 268A and a first ferrule 272A and/or a second articulation member 268B and a second ferrule 272B in a straight configuration (e.g., parallel to longitudinal axis B). Core 262 may also be configured to orient first ferrule 272A against a first outermost wall 276A and second ferrule 272B against a second outermost wall 276B of core 262. As such, an internal diameter of a lumen 278 (e.g., a central lumen) may receive cabling and/or tubes of insertion portion 214.

Core 262 may further include a first channel 270A configured to receive a portion of first articulation member 268A (e.g., proximal of first ferrule 272A). A second channel 270B may be configured to receive a portion of second articulation member 268B (e.g., proximal of second ferrule 272B). A proximal end of first ferrule 272A may abut a first distal face 274A of first channel 270A. Similarly, a proximal end of a second ferrule 272B may abut a second distal face 274B of second channel 270B. Each of first distal face 274A and second distal face 274B may be approximately perpendicular to a longitudinal axis A of core 262. As such, first ferrule 272A, second ferrule 272B, and a distal end of each of first articulation member 268A and second articulation member 268B may be approximately parallel to longitudinal axis A. Core 262 may be configured such that a distal end of each of first articulation member 268A, first ferrule 272A, second articulation member 268B, and second ferrule 272B terminates proximal to a distalmost end of core 262.

Each of first channel 270A and second channel 270B may be offset from a center of a first distal face 274A and second distal face 274B, respectively. For example, a first wall 271A forming first channel 270A may be continuous with first outermost wall 276A, and a second wall 271B forming second channel 270B may be continuous with second outermost wall 276B. First channel 270A may be further defined by a first internal wall 273A. For example, a distal end of first internal wall 273A may comprise first distal face 274A. In such a way, the proximal face of first ferrule 272A may abut the distal face (e.g., first distal face 274A) of first internal wall 273A. Accordingly, only a portion of the proximal face of first ferrule 272A may be configured to abut first distal face 274A. The remaining portion of the proximal face of first ferrule 272A may extend over a distal opening of first channel 270A. Similarly, second channel 270B may be further defined by a second internal wall 273B. A proximal face of second ferrule 272B may be configured to abut a distal face (e.g., second distal face 274B) of second internal wall 273B. As such, only a portion of the proximal face of first ferrule may be configured to abut first distal face 274A. The remaining portion of the proximal face of second ferrule 272B may extend over a distal opening of second channel 270B.

A distal portion (e.g., distal to each of first internal wall 273A and second internal wall 273B) of central lumen 278 may be circular or ovular. A proximal portion of central lumen 278 (e.g., between a proximal end and a distal end of each off first internal wall 273A and second internal wall 273B) may resemble a figure eight shape. For example, a cross-sectional shape of the proximal portion of central lumen 278 may be similar to that of FIG. 2B. For example, a first diameter D1 of central lumen 278 may be smaller than a second diameter D2 and/or a third diameter D3. First diameter D1 may be smaller, for example, due to first internal wall 273A and second internal wall 273, opposite of first internal wall 273A, protruding into central lumen 278.

In some aspects, each of first articulation member 268A and second articulation member 268B may be loosely contained within first channel 270A and second channel 270B, respectively. For example, each of first articulation member 268A and second articulation member 268B may be movable (e.g., rotatable) within first channel 270A and second channel 270B, respectively. In other aspects, first articulation member 268A and/or second articulation member 268B may be fixed within each respective channel, for example, via an adhesive. Additionally or alternatively, first articulation member 268A and second articulation member 268B may be overmolded or otherwise fixed within core 262. Accordingly, with first ferrule 272A and second ferrule 272B oriented parallel to longitudinal axis B of core 262, additional space may be created within distal tip 244 (e.g., distal to core 262). The additional space may within distal tip 244 may be utilized, for example, to accommodate additional components (e.g., imaging devices, light sources sensors, etc.).

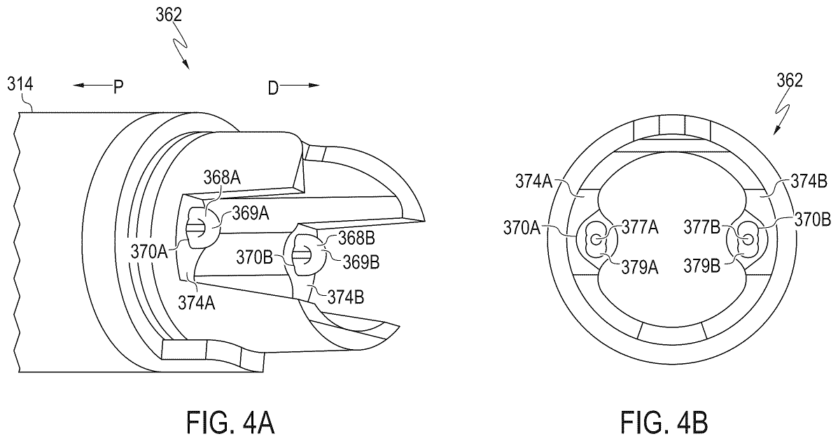

FIGS. 4A and 4B illustrate another example according to the present disclosure, showing core 362. FIG. 4A shows a perspective view of core 362 coupled to an insertion portion 314. FIG. 4B illustrates a front, or top-down, view of core 362. Core 362 may have any or all of the characteristics of core 162 and/or core 262, except as described below. FIGS. 4A and 4B illustrate an example wherein articulation members are fixed to a core of a distal tip without ferrules or crimped portions. For example, core 362 may include a first channel 370A configured to receive a first articulation member 368A and a second channel 370B configured to receive a second articulation member 368B. First articulation member 368A may be configured to form a first bend 369A and second articulation member 368B may be configured to form a second bend 369B. Each of first bend 369A and second bend 369B may be distal to a first face 374A and a second face 374B of core 362, respectively.

As shown in FIG. 4B, the cross-sectional size and/or shape of each of first channel 370A and second channel 370B may vary. For example, a first proximal portion 377A of first channel 370A may have a smaller cross-sectional dimension than a first distal portion 379A of first channel 370A. Further, first proximal portion 377A may have a cross-sectional dimension that is circular, square, polygonal, etc. First proximal portion 377A may be configured to receive first articulation member 368A. First distal portion 379A of first channel 370A may have a greater cross-sectional dimension than first proximal portion 377A. First distal portion 377A may be configured to receive at least two portions of first articulation member 368A, e.g., a portion of first articulation member 368A just before and just after first bend 369A. In some aspects, first distal portion 379A may have a diameter that is twice as great as a diameter of first proximal portion 377A.

In some aspects, each of first articulation member 368A and second articulation member 368B may be fixed to core 362, for example, with an adhesive. Additionally or alternatively, first articulation member 368A with first bend 369A and second articulation member 368B with second bend 369B may be overmolded or otherwise fixed within core 362.

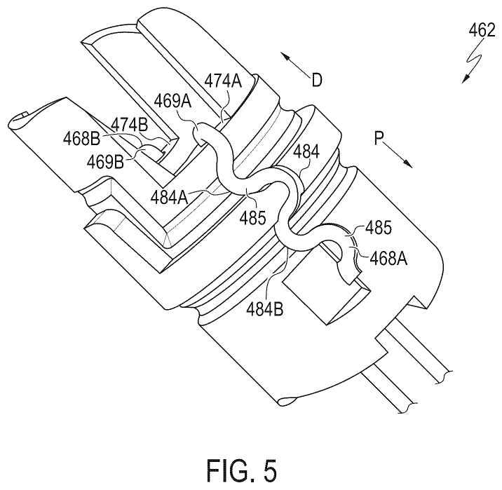

FIG. 5 illustrates a perspective view of another exemplary core 462 of a distal tip of a medical device according to the present disclosure. Core 462 may have any or all of the characteristics of core 162, core 262, and/or core 362, except as described below. Core 462 may be configured to receive a first articulation member 468A via a first channel extending longitudinally through core 462, and a second articulation member 568B via a second channel extending longitudinally through core 462. First articulation member 468A may include a first distal bend 469A distal to a first distal surface 474A of core 462. Similarly, second articulation member 468B may include a second distal bend 469B distal to a second distal surface 474B of core 462.

Core 462 may include a groove 484 having a plurality of curves 484A extending proximally along one or more external, or outside, surfaces of core 462. Groove 484 may be configured to receive first articulation member 468A (e.g., a portion of first articulation member 468A that is after first bend 469A). In some aspects, first articulation member 468A may be fixed to core 562 within groove 484, e.g., via an adhesive. Additionally or alternatively, first articulation member 468A may be overmolded within core 462, e.g., such that a distal portion of first articulation member 468A (e.g., after first bend 469A) may be at least partially embedded within core 462.

Core 462 may include a second groove having a second plurality of curves on an opposite side of core 462 (e.g., a side of core 462 facing into the page). The second groove may similarly extend along an external, or outside, surface of core 462. The second groove may be configured to similarly receive second articulation member 468B. In some aspects, the second groove may similarly fix second articulation member 468B to core 462, optionally with an adhesive or via overmolding.

Each of first articulation member 468A and second articulation member 468B may be fixed to core 462, for example, without a ferrule or a crimp fixed to each articulation wire.

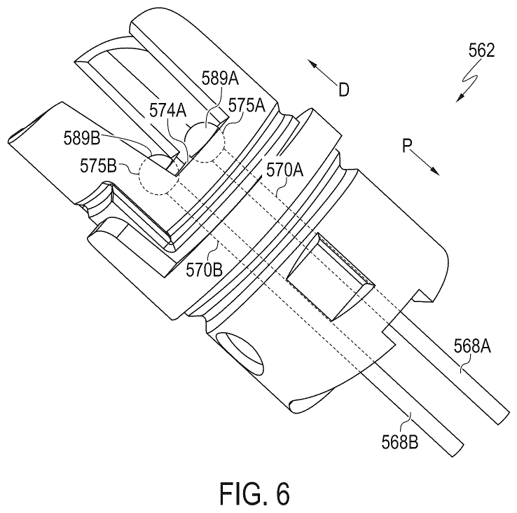

FIG. 6 illustrates a perspective view of another exemplary core 562 of a distal portion of a medical device according to the present disclosure. Core 562 may have any or all of the characteristics of core 162, core 262, core 362, and/or core 462 unless otherwise specified.

Core 562 may be configured to receive one or more articulation members extending longitudinally through core 562. For example, core 562 may include a first channel 570A and a second channel 570B each extending longitudinally through core 562. First channel 570A and second channel 570B may be configured to receive a first articulation member 568A and a second articulation member 568B, respectively. First articulation member 568A may include a first distal member 589A fixed or coupled to a distalmost end of first articulation member 568A and second articulation member 568B may include a second distal member 589B fixed to a distalmost end of second articulation member 568B. Each of first distal member 586A and second distal member 589B may be formed in the shape of a sphere, or a ball, although other shapes such as, e.g., diamond shaped, square shaped, etc., may be used.

In some aspects, a first distal face 574A of core 562 may include a first indentation 575A configured to receive (e.g., have a size complementary to) first distal member 589A of first articulation member 568A. A second distal face 574B of core 562 may include a second indentation 575B configured to receive second distal member 586B of second articulation member 568B. Each of first indentation 575A and second indentation 575B may be configured such that, for example, each of first distal member 586A and second distal member 586B extends distally past first distal face 574A and second distal face 574B, respectively.

In some aspects, each of first articulation member 568A and second articulation member 568B may be loosely contained within first channel 570A and second channel 570B, respectively. In other aspects, an adhesive may be used to fix first articulation member 568A and second articulation member 568B to core 562 and/or first distal member 586A and second distal member 586B may be at least partially overmolded within core 562.

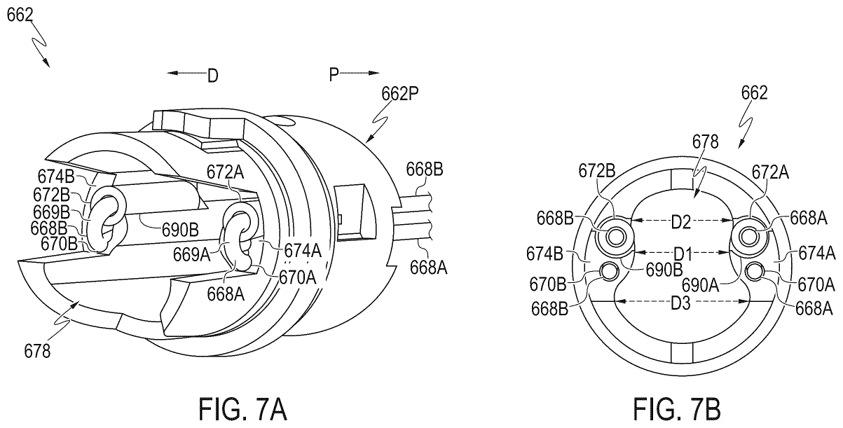

FIGS. 7A and 7B illustrate another example of a core 662 of a distal tip of a medical device according to the present disclosure. FIG. 7A illustrates a perspective view of core 662 with a first articulation member 668A and a second articulation member 668B. Core 662 may include a first channel 670A configured to receive a first articulation member 668A and a first pocket 690A configured to receive a first ferrule 672A, e.g., fixed to a distal end of first articulation member 668A. First pocket 690A may be adjacent to at least a portion of first channel 670A. For example, first pocket 690A may have a longitudinal length shorter than that of first channel 670A. In some aspects, first channel 670A may be separate from first pocket 690A and from a central lumen 678 of core 662. At least a portion of first pocket 690A may be open to, or in communication with, a lumen 678.

Core 662 may further include a second channel 670B configured to receive a second articulation member 668B and a second pocket 690B configured to receive a second ferrule 672B, similar to the features of first articulation member 668A.

Each of first pocket 690A and second pocket 690B may be parallel to lumen 678. In such a way, first ferrule 672A and second ferrule 672B may be parallel to lumen 678. In other examples, first pocket 690A and/or second pocket 690B may be configured so as to angle first ferrule 672A and/or second ferrule 627B inwards (e.g., towards a longitudinal axis extending proximally and distally through core 662). In some aspects, each of first pocket 690A and second pocket 690B may extend proximally through a portion of core 662. As such, each of first ferrule 672A and second ferrule 672B may be fully contained within core 662. For example, core 662 may be configured such that at least a portion of one or both of first ferrule 672A and second ferrule 672B is distal to a proximalmost face 662P of core 662.

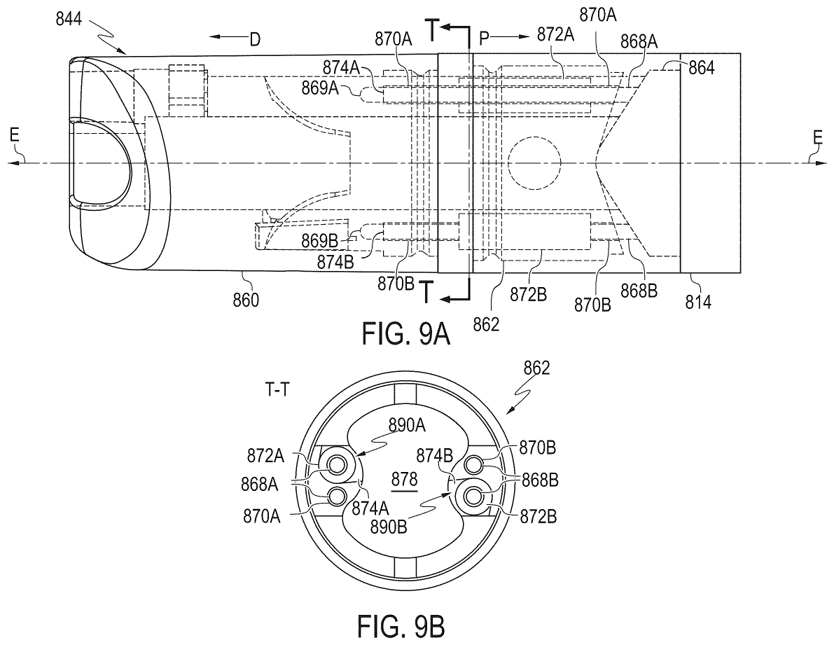

Each of first articulation member 668A and second articulation member 668B may be configured to bend approximately 180 degrees. For example, first articulation member 668A may be configured to form a first bend 669A. Second articulation member 668B may be configured to form a second bend 669B. Each of first bend 669A and second bend 669B may be distal to a first face 674A and a second face 674B of core 662, respectively. Each of first bend 669A and second bend 669B may be in the same direction (e.g., towards a side of the page). In other examples, each of first bend 669A and second bend 669B may be in different directions (e.g., see FIGS. 9A and 9B, discussed below).

A portion of lumen 678 that includes first channel 670A, first pocket 690A, second channel 270B, and second pocket 690B may have a first diameter D1, a second diameter D2, and a third diameter D3, wherein second diameter D2 may be smaller than first diameter D1 and third diameter D3. For example, first pocket 690A and second pocket 690B each have a larger cross-sectional diameter than the cross-sectional diameters of first channel 670A and second channel 670B. Third diameter D3 may be greater than first diameter D1 and second diameter D2. First diameter D1 may be greater than second diameter D2, but smaller than third diameter D3.

Core 662 may be configured such that a portion of each of first articulation member 668A and second articulation member 668B is bent proximally within core 662. With first articulation member 668A bent proximally, first ferrule 672A may be proximal relative to the distalmost end of core 662. Accordingly, additional space may be created within the distal tip (e.g., distal to core 662). Similarly, with second articulation member 668B bent proximally, second ferrule 672B may be proximal relative to the distalmost end of core 662. With second ferrule 672B oriented in such a way, additional space may be created within the distal tip (e.g., distal to core 662). For example, first pocket 690A may have a smaller longitudinal length than first channel 670A. The additional space within the distal tip may allow for distal tip 144 to accommodate additional components (e.g., imaging devices, light sources, sensors, etc.).

Additionally or alternatively, with first articulation member 668A and second articulation member 668B bent proximally, tensile forces being applied to each respective ferrule (e.g., first ferrule 672A and second ferrule 672B) may be decreased. In some aspects, the first bend 669A and second bend 669B may decrease the likelihood of separation between each respective articulation member and distal member (e.g., separation of first articulation member 668A from first ferrule 672A or second articulation member 668B from second ferrule 672B) and/or the separation of each articulation member from core 662 (e.g., one or both of first articulation member 668A and second articulation member 668B from core 662).

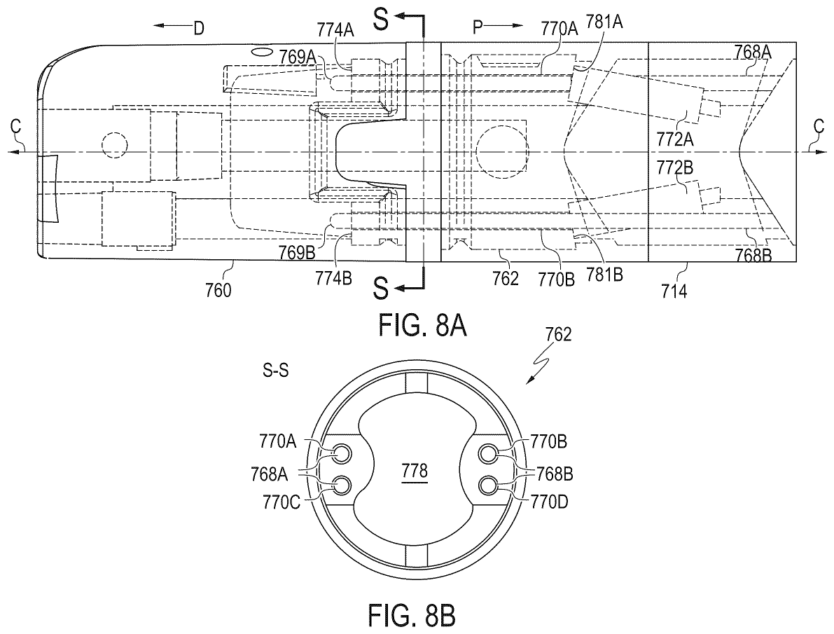

FIG. 8A illustrates a side view of another exemplary core 762, for example, of a medical device that includes insertion portion 714. Dashed lines are used to illustrate internal components of, or components contained within, insertion portion 714 and/or a cap 760. FIG. 8B illustrates a cross-sectional view of the distal portion of insertion portion 714 taken at cross-sectional plane S-S. Insertion portion 714 may have any or all of the characteristics of insertion portion 14, 114, 214, and/or 314. The distal portion of insertion portion 714 may include a cap 760 and a core 762. Cap 760 may have any or all of the characteristics of any cap previously described (e.g., cap 160, 260). Core 762 may have any or all of the characteristics of any core previously described (e.g., core 162, 262, 362, 462, 562, 662), except as described below. For example, core 762 may be configured to receive a first articulation member 768A via a first channel 770A. Core 762 may additionally be configured to receive a second articulation member 768B via a second channel 770B. Each of first articulation member 768A and second articulation member 768B may be configured to bend approximately 180 degrees. For example, first articulation member 768A may include a first distal bend 769A distal to a first distal surface 774A of core 762. Similarly, second articulation member 768B may include a second distal bend 769B distal to a second distal surface 774B of core 762.

Core 762 may further include a third channel 770C and a fourth channel 770D. Third channel 770C may extend adjacent to and/or parallel to first channel 770A, for example, within core 762. Similarly, fourth channel 770D may extend adjacent to and/or parallel to second channel 770B. Third channel 770C may be configured to receive a second portion of first articulation member 768A (e.g., a portion of first articulation member 768A that is after first bend 769A). Fourth channel 770D may be configured to receive a second portion of second articulation member 768B (e.g., a portion of second articulation member 768B that is after second bend 769B). Each of first channel 770A, second channel 770B, third channel 770C, and fourth channel 770D may be parallel and adjacent to a lumen 778 of core 762. Lumen 778 may have a similar cross-sectional shape as described with reference to lumen 178 of FIG. 2B and/or lumen 278 of FIG. 3B.

A first ferrule 772A may be fixed to a distal end of first articulation member 768A. A second ferrule 772B may be fixed to a distal end of second articulation member 768B. A distal face of each of first ferrule 772A may abut a first proximalmost face 781A of core 762. Similarly, a distal face of second ferrule 772B may abut a second proximalmost face 781B of core 762. In such a way, at least a portion of each of first ferrule 772A and second ferrule 772B may be proximal of core 762. In some aspects, at least a portion of first ferrule 772A and second ferrule 772B may extend proximally into, or through, one or more articulation links 764.

Each of first proximalmost face 781A and second proximalmost face 781B of core 762 may be angled relative to one another and relative to a longitudinal axis C. First proximalmost face 781A may extend along a first plane transverse to longitudinal axis C of core 762. Second proximalmost face 781B may extend along a second plane transverse to longitudinal axis C of core 762. For example, first proximalmost face 781A and second proximalmost face 781B may be angled such that an outermost edge (e.g., an edge farthest away from longitudinal axis A) of each of first proximalmost face 781A and second proximalmost face 781B is proximal relative to an innermost edge (e.g., an edge closes to longitudinal axis A) of each of first proximalmost face 781A and second proximalmost face 781B.

With each of first proximalmost face 781A and second proximalmost face 781B angled, first ferrule 772A and second ferrule 772B may be similarly angled. For example, each of first ferrule 772A and second ferrule 772B may be angled inwards (e.g., towards longitudinal axis A). Furthermore, core 762 may be configured such that each of first ferrule 772A and second ferrule 772B is proximal of core 762 (e.g., proximal of first proximalmost face 781A and second proximalmost face 781B) With each of first ferrule 772A and second ferrule 772B oriented in such a way, additional space may be created within the distal tip (e.g., distal to core 762). Additionally or alternatively, with each of first articulation member 768A, first ferrule 772A, second articulation member 768B, and second ferrule 772B oriented in such a way, tensile forces being applied to each respective ferrule (e.g., first ferrule 772A and/or second ferrule 772B) may be decreased. As such, the likelihood of separation between the components and/or from core 762 may be decreased.

FIG. 9A illustrates a side view of another exemplary core 862 of a distal tip 844 of an insertion portion 814. FIG. 9B illustrates a cross-sectional view of core 862 taken at cross-section T-T of FIG. 9A. Insertion portion 814 may have any or all of the characteristics of the insertion portions previously discussed. For example, the distal portion of insertion portion 814 may include distal tip 844. Distal tip 844 may include a cap 860 and a core 862. Core 862 may be distal to one or more articulation joints 864. Dashed lines are used to illustrate internal components of, or components contained within, the distal portion of insertion portion 814. For example, core 862 is shown in dashed lines in FIG. 9A because core 862 is contained within insertion portion 814.

Core 862 may have any or all of the characteristics of any of the cores previously discussed (e.g., core 162, 262, 362, 462, 562, 662, 762), except as described below. For example, core 862 may be configured to receive first articulation member 868A via a first channel 870A and second articulation member 868B via a second channel 870B. First articulation member 868A and first articulation member 868A may be configured to bend approximately 180 degrees in opposite directions. For example, first articulation member 868A may include a first distal bend 869A that is bent in a first direction. First distal bend 869A may be distal to a first distal surface 874A of core 862. Similarly, second articulation member 868B may include a second distal bend 869B that is bent is a second direction, opposite the first. Second distal bend 869B may be distal to a second distal surface 874B of core 862.

A first ferrule 872A may be fixed to a distal end of first articulation member 868A. A second ferrule 872B may be fixed to a distal end of second articulation member 868B. Core 862 may further include a first pocket 890A configured to receive first ferrule 872A and a second pocket 890B configured to receive second ferrule 872B. Each of first pocket 890A and second pocket 890B may be parallel and/or adjacent to each respective channel (e.g., first channel 870A and/or second channel 870B). Similarly, each of first pocket 890A and second pocket 890B may be parallel to a longitudinal axis E of distal tip 844. A distal end of first pocket 890A may be defined by a first proximal face 881A. A distal end of second pocket 890B may be defined by a second proximal face 881B. As such, with first ferrule 872A received within first pocket 890A, a proximal face of first ferrule 872A may abut, or touch, first proximal face 881A of first pocket 890A. Similarly, with second ferrule 872B received within second pocket 890B, a proximal face of second ferrule 872B may abut, or touch, second proximal face 881B of second pocket 890B.

Core 862 may be configured such that a portion of each of first articulation member 868A and second articulation member 868B may be bent and fixed in opposite directions within core 862. For example, first articulation member 868A and/or second articulation member 868B may be bent proximally by approximately 180 degrees in opposite directions. With first articulation member 868A and second articulation member 868B bent proximally in opposite directions, each of first ferrule 872A and second ferrule 872B may be proximal relative to the distalmost end of core 862 and distal relative to a proximalmost end of core 862. In such a way, additional space may be created within the distal tip (e.g., distal to core 862). The additional space within the distal tip 844 may allow for distal tip 844 to accommodate additional components (e.g., additional imaging devices, light sources, tubes, sensors, etc.). Additionally or alternatively, with each of first articulation member 868A, first ferrule 872A, second articulation member 868B, and second ferrule 872B oriented in such a way, tensile forces being applied to each respective ferrule (e.g., first ferrule 872A and/or second ferrule 872B) may be decreased. As such, the likelihood of separation between the components and/or from core 862 may be decreased.

Furthermore, with this configuration, a cross-sectional shape of a lumen 878 may be maximized. For example, the cross-sectional shape of central lumen 878 may be similar to the cross-sectional shape of lumen 178, discussed above with respect to FIG. 2B, or lumen 278, discussed above with respect to FIG. 3B. Specifically, the cross-sectional shape of central lumen 878 may resemble a figure eight.

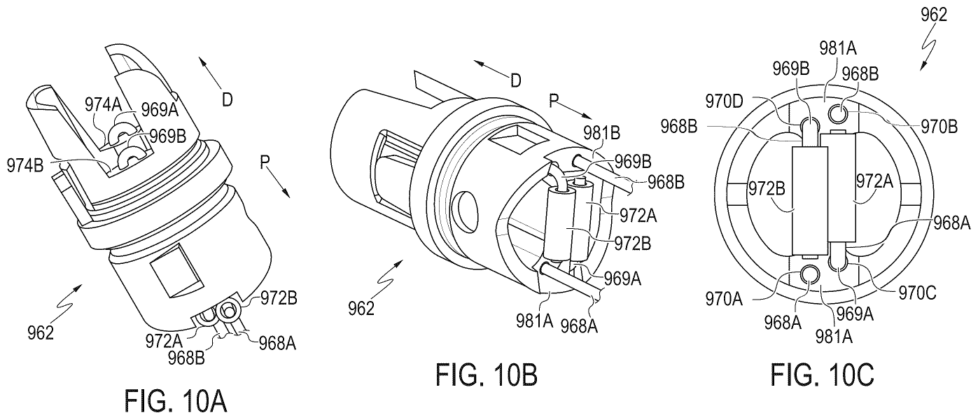

FIGS. 10A-10C depict another core 962 according to the present disclosure with a first articulation member 968A and a second articulation member 968B. FIG. 10A illustrates a side perspective view of core 962; FIG. 10B illustrates a back perspective view of core 962; and FIG. 10C illustrates a back view of core 962. Core 962 may have any or all of the characteristics of any core previously discussed, except as described below. For example, the distal portion of any insertion portion previously discussed (e.g., insertion portion 14, 114, 214, 314, 714, 814) may include core 962. Similarly, any cap previously discussed (e.g., cap 160, 260, 760, 860) may be coupled to core 962.

Core 962 may be configured to receive a first portion of first articulation member 968A via a first channel 970A. Core 962 may be configured to receive a first portion of second articulation member 968B via a second channel 970B. Each of first channel 970A and second channel 970B may extend longitudinally through at least a portion of core 962. For example, a proximal opening of each of first channel 970A and second channel 970B may be on a first proximal face 981A and a second proximal face 981B, respectively. A distal opening of each of first channel 970A and second channel 970B may be on a first distal face 974A and a second distal face 974B, respectively. Each of first channel 910A and second channel 970B may extend through core 962, for example, parallel to a central lumen 978 of core 962.

Each of first articulation member 968A and second articulation member 968B may be configured to bend approximately 180 degrees. For example, first articulation member 968A may include a first distal bend 969A. First distal bend 969A may be distal to first distal face 974A of core 962. Similarly, second articulation member 968B may include a second distal bend 969B. Second distal bend 969B may be distal to second distal face 974B of core 962. First bend 969A and second bend 969B may be in opposite directions.

Core 962 may further include a third channel 970C and a fourth channel 970D. Third channel 970C may extend adjacent to and/or parallel to first channel 970A, for example, within core 962. Similarly, fourth channel 970D may extend adjacent to and/or parallel to second channel 970B. Third channel 970C may be configured to receive a second portion of first articulation member 968A (e.g., a portion of first articulation member 968A that is after first bend 969A). Fourth channel 970D may be configured to receive a second portion of second articulation member 968B (e.g., a portion of second articulation member 968B that is after second bend 969B). Each of third channel 970C, and fourth channel 970D may be parallel and adjacent to central lumen 978 of core 962.

In some aspects, a distal end of each of first articulation member 968A and second articulation member 968B may extend proximally past first proximal face 981A and second proximal face 981B, respectively. A first ferrule 972A may be fixed to a distal end of first articulation member 968A. A second ferrule 972B may be fixed to a distal end of second articulation member 968B. First articulation member 968A may include a third bend 969C. Third bend 969C of first articulation member 968A may be just before first ferrule 972A. Third bend 969C may be in a direction that is perpendicular to first bend 969A. As such, third bend 969C may be configured to orient, or position, first ferrule 972A within and/or across lumen 978 (e.g., perpendicular to a longitudinal axis of core 962).

Second articulation member 968B may include a fourth bend 969D. Fourth bend 969D may be just before second ferrule 972B. Fourth bend 969D may be in a direction that is perpendicular to second bend 969B. In some aspects, fourth bend 969D may be in an opposite direction as third bend 969C. As such, fourth bend 969D may be configured to orient, or position, second ferrule 972B within and/or across central lumen 978 such that second ferrule 972B is perpendicular to the longitudinal axis of core 962. Accordingly, first ferrule 972A and second ferrule 972B may be parallel to one another. First ferrule 972A may extend in an opposite direction than second ferrule 972B.

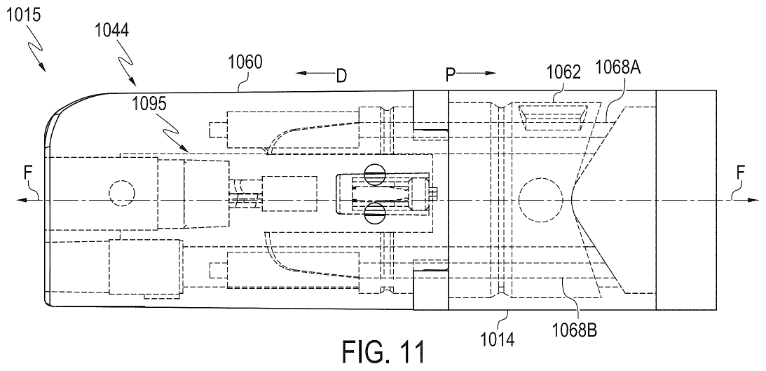

FIG. 11 illustrates an alternative assembly of a distal portion of an insertion portion 1014. Dashed lines are used to illustrate internal components of, or components contained within, insertion portion 1014. The distal portion of insertion portion 1014 may have any or all of the characteristics of any insertion portion previously discussed (e.g., insertion portion 14, 114, 214, 314, 714, 814), except as described. In this embodiment, internal components of the distal portion of insertion portion 1014 may be rearranged, for example, in a linear assembly 1095. Linear assembly 1095 may include an imaging device 1052 and/or one or more light sources 1050. Linear assembly 1095 may be configured, for example, to create additional space within the distal portion of insertion portion 1014 (e.g., radially around linear assembly 1095). In some aspects, linear assembly 1095 may be easy to assembly, for example, due to the additional space created within the distal portion (e.g., when linear assembly 1095 is utilized).

A distal tip 1044 of insertion portion 1014 may include a cap 1060 and a core 1062. Core 1062 may be configured to receive a first articulation member 1068A and/or a second articulation member 1068B. For example, first articulation member 1068A and second articulation member 1068B may extend longitudinally through core 1062 (e.g., via one or more channels of core 1062). In some aspects, each of first articulation member 1068A and second articulation member 1068B may be parallel to a longitudinal axis F of insertion portion 1014. Each of first articulation member 1068A and second articulation member 1068B may extend at least partially along either side of linear assembly 1095, for example, parallel to a longitudinal axis F extending through distal tip 1044. Although not shown, core 1062 may include additional channels, or lumens, for example, configured to receive one or more wires, cables, sensors, etc., associated with linear assembly 1095 and/or other aspects of the distal portion of insertion portion 1014.

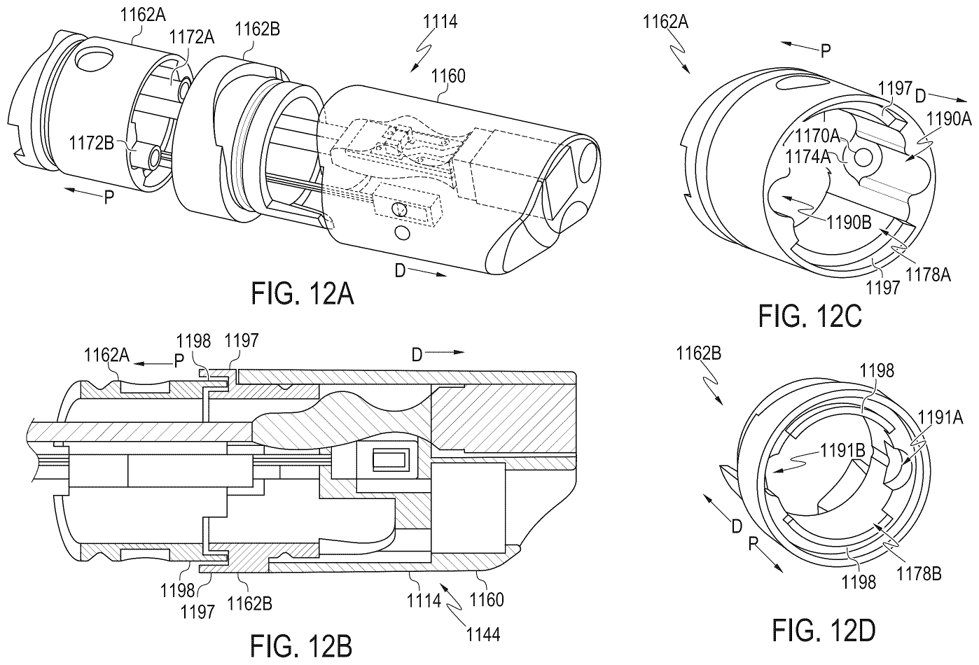

FIGS. 12A-12D illustrate various views of another assembly of a distal portion of insertion portion 1114 comprising at least a cap 1160, a first core portion 1162A and a second core portion 1162B. For example, FIG. 12A illustrates an exploded view of the distal portion of insertion portion 1114; FIG. 12B illustrates an assembled cross-section of the distal portion of insertion portion 1114; FIG. 12C illustrates a perspective view of first core portion 1162A; and FIG. 12D illustrates a perspective view of the second core portion 1162B. Accordingly, elements or features of first core portion 1162A and/or second core portion 1162B may be observed in one or more figures of FIGS. 12A-12D.

In some aspects, first core portion 1162A and second core portion 1162B may be configured to couple together, for example, via a screw fit. Second core portion 1162B may be configured to couple to cap 1160, for example, via a press-fit. First core portion 1162A may be proximal of second core portion 1162B and cap 1160, for example, in an assembled state (e.g., see FIG. 12B). As such, a proximal portion of second core portion 1162B may be configured to receive a distal portion of first core portion 1162A. A proximal portion of cap 1160 may be configured to receive a distal portion of second core portion 1162B.

Each of first core portion 1162A and second core portion 1162B may be configured to receive at least a portion of a first articulation member 1168A and second articulation member 1168B. For example, first core portion 1162A may include a first channel 1170A, shown in FIG. 12C. First channel 1170A may be configured to receive a portion of first articulation member 1168A (e.g., the portion of first articulation member 1168A that is proximal of a first ferrule 1172A). Although not shown, first core portion 1162A may further include a second channel, for example, on an opposite side of a first central lumen 1178A of first core portion 1162A. The second channel may be configured to receive a portion of the second articulation member (e.g., the portion of the second articulation member that is proximal of second ferrule 1172B).

First core portion 1162A may include a first pocket 1190A. First pocket 1190A may be configured to receive first ferrule 1172A of the first articulation member 1168A. In some aspects, a proximal face of first ferrule 1172A may be configured to abut, or touch, a first distal face 1174A defining a proximal end of first pocket 1190A. First pocket 1190A may be open to the first lumen 1178A of first core portion 1162A.

First distal face 1174A may be perpendicular relative to a longitudinal axis of first core portion 1162A. As such, when first ferrule 1172A is seated within first pocket 1190A (e.g., such that the proximal end of first ferrule 1172A abuts first distal face 1174A), first ferrule 1172A and first articulation member 1168A may be aligned parallel to a longitudinal axis of first core portion 1162A. In alternative examples, first distal face 1174A may be angled relative to the longitudinal axis of first core portion 1162A. As such, when first ferrule 1172A is seated within first pocket 1190A (e.g., such that the proximal end of first ferrule 1172A abuts first distal face 1174A), first ferrule 1172A and first articulation member 1168A may be angled towards the longitudinal axis of first core portion 1162A.

First core portion 1162A may further include a second pocket 1190B configured to receive second ferrule 1172B. In some aspects second pocket 1190B may be on an opposite side of first central lumen 1178A, for example, directly opposite first pocket 1190A. Although not shown, second pocket 1190B may have any or all the same aspects of first pocket 1190A. For example, second pocket 1190B may be open to first central lumen 1178A. Second pocket 1190B may include a second distal face defining a proximal end of second pocket 1190B. Second ferrule 1172B may be configured to abut, or touch, the second distal face. In some aspects, second distal face may be approximately perpendicular to the longitudinal axis of first core portion 1162A such that second ferrule 1172B is parallel relative to the longitudinal axis when seated within second pocket 1190B. In other aspects, second distal face of second pocket 1190B may be angled, for example, to angle second ferrule 1172B inwards (e.g., towards the longitudinal axis of first core portion 1162A.

A distal end of first core portion 1162A may include one or more coupling features 1197. A proximal end of second core portion 1162B may include one or more complementary coupling features 1198. In some aspects, coupling feature(s) 1197 of first core portion 1162A may include a plurality of internal threads, for example, to engage with a plurality of external threads (e.g., complementary coupling features 1198) disposed on the proximal end of second core portion 1162B. As such, first core portion 1162A and second core portion 1162B may be configured to couple via a screw fit. In other examples, coupling feature(s) 1197 of first core portion 1162A may include a lip or a groove, and coupling feature(s) 1198 of second core portion 1162B may include a complementary groove or lip. As such, first core portion 1162A and second core portion 1162B may be configured to couple via a snap-fit or a press-fit. Additionally or alternatively, first core portion 1162A and second core portion 1162B may be coupled via a bonding substance (e.g., a glue, an adhesive, an epoxy, etc.), or without the need of coupling features 1197 or complementary coupling features 1198.

Second core portion 1162B may further include a first receiving pocket 1191A. First receiving pocket 1191A of second core portion 1162B may be configured to align with first pocket 1190A of first core portion 1162A, for example, when first core portion 1162A and second core portion 1162B are coupled together. First receiving pocket 1191A may be open to a central lumen 1178B of second core portion 1162B.

First receiving pocket 1191A may be configured to receive a distal portion of first ferrule 1172A. For example, a distal face of first ferrule 1172A may abut, or touch, a first proximal face 1192A defining a distal end of first receiving pocket 1191A. In such a way, for example, when first core portion 1162A is fixed to second core portion 1162B, first ferrule 1172A may be fixed between first distal face 1174A of first pocket 1190A (see FIG. 12C) and first proximal face 1192A of first receiving pocket 1191A of second core portion 1162B. As such, first ferrule 1172A may be fixed within the distal portion, thus fixing first articulation member 1168A within distal tip 1144. In some aspects, first ferrule 1172A may be fixed so as to not be able to move longitudinally within the distal portion, but still be allowed to rotate within first core portion 1162A and second core portion 1162B.

Second core portion 1162B may further include a second receiving pocket 1191B. Second receiving pocket 1191B may be configured to receive a distal portion of second ferrule 1172B. As such, second receiving pocket 1191B may have any or all of the same characteristics as first receiving pocket 1191A. For example, although not shown, a distal face of second ferrule 1172B may abut, or touch, a second proximal face of defining a distal end of second receiving pocket 1191B. In such a way, for example, when first core portion 1162A is fixed to second core portion 1162B, second ferrule 1172B may be fixed between the second distal face of second pocket 1190B and the proximal face of second receiving pocket 1191B. As such, second ferrule 1172B may be fixed within the distal portion, thus fixing second articulation member 1168B within distal tip 1144. In some aspects, second ferrule 1172B may be fixed so as to not be able to move longitudinally within the distal portion, but still be allowed to rotate within first core portion 1162A and second core portion 1162B.

In some aspects, first core portion 1162A and second core portion 1162B may be configured, for example, so as to contain, or confine, each of first articulation member 1168A and second articulation member 1168B within a proximal portion of distal tip 1144 of insertion portion 1114. As such, additional space within the distal portion may be created (e.g., distal to second core portion 1162B).

Additionally or alternatively, first core portion 1162A and second core portion 1162B may be configured, for example, so as to minimize, or reduce the likelihood, of damage to the internal components of the distal portion. For example, because each of first articulation member 1168A and second articulation member 1168B are each confined between first core portion 1162A and second core portion 1162B, first articulation member 1168A and second articulation member 1168B are prevented from catching, or snagging, the internal components (e.g., the wiring or cabling of the lighting elements and/or the imaging elements of the distal portion).

Each of the embodiments discussed herein are exemplary. For example, the exemplary embodiments discussed herein provide for various alternative configurations of one or more articulation members and/or electronic assemblies within the distal portion of an insertion portion of the medical devices. The various configurations may be utilized, for example, such that the articulation member(s) occupy a relatively small, or limited, amount of space within the distal tip, while retaining quality of performance and manufacturing. The additional space created with such configurations may be utilized, for example, to accommodate additional components and/or assist in making the distal portion of the medical device easier to assemble. In some aspects, the configurations discussed herein may have a reduced cross-sectional size. Additionally or alternatively, the overall length of the configurations of the distal tips discussed herein may be reduced.

While principles of this disclosure are described herein with the reference to illustrative examples in a particular context and for particular medical procedures, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

1. A medical device comprising:
a handle;
an insertion portion extending distally from the handle, wherein a first articulation member and a second articulation member each extend longitudinally from the handle through the insertion portion, and wherein the insertion portion defines a first lumen; and
a distal tip at a distal end of the insertion portion, wherein the distal tip includes:
a light source;
an imaging device; and
a core including a first channel that receives a distal portion of the first articulation member, a second channel that receives a distal portion of the second articulation member, and a second lumen between the first channel and the second channel, the second lumen being in communication with the first lumen of the insertion portion;
wherein each of the first articulation member and the second articulation member terminates proximal to a distalmost end of the core; and
wherein the first articulation member includes a first bend distal to a first distal face defining a distal end of the first channel, and the second articulation member includes a second bend distal to a second distal face defining a distal end of the second channel.

2. The medical device of claim 1, further comprising:
a first distal member fixed to the distal portion of the first articulation member, wherein a proximal face of the first distal member abuts the first distal face of the core; and
a second distal member fixed to a distal end of the second articulation member, wherein a proximal face of the second distal member abuts the second distal face of the core.

3. The medical device of claim 1, wherein each of the first distal face and the second distal face of the core extends along a plane transverse to a longitudinal axis of the core.

4. The medical device of claim 1, wherein each of the first bend and the second bend is approximately 90 degrees to approximately 180 degrees.

5. The medical device of claim 1, wherein the first bend is in a first direction, and the second bend is in a second direction, opposite the first direction.

6. The medical device of claim 5, wherein the core further includes:
a first groove on an external surface of the core that receives the distal portion of the first articulation member; and
a second groove on an external surface of the core that receives the distal portion of the second articulation member.

7. The medical device of claim 6, wherein each of the light source and the imaging device is distal to the distal portion of each of the first articulation member and the second articulation member.

8. The medical device of claim 1, wherein the core further includes:
a first pocket adjacent to the first channel, wherein the first pocket is in communication with the first lumen of the core and receives a first distal member fixed to the distal portion of the first articulation member; and
a second pocket adjacent to the second channel, wherein the second pocket is in communication with the second lumen of the core and receives a second distal member fixed to the distal portion of the second articulation member.

9. The medical device of claim 8, wherein a proximal face of the first distal member of the first articulation member abuts a distal end of the first pocket, and a proximal face of the second distal member of the second articulation member abuts a distal end of the second pocket.

10. The medical device of claim 1, wherein the core further includes:
a third channel adjacent to the first channel, wherein the third channel receives a portion of the first articulation member after the first bend; and
a fourth channel adjacent to the second channel, wherein the fourth channel receives a portion of the second articulation member after the first bend.

11. The medical device of claim 2, wherein a proximal face of the first distal member abuts a first proximal face of the core that defines a proximal end of the first channel, and a proximal face of the second distal member abuts a second proximal face of the core that defines a proximal end of the second channel.

12. The medical device of claim 11, wherein each of the first proximal face and the second proximal face extends along a plane transverse to a longitudinal axis of the core.

13. The medical device of claim 11, wherein the first articulation member further includes a third bend proximal to the first proximal face of the core, wherein the third bend is in a third direction perpendicular to the first bend, and
wherein the second articulation member further includes a fourth bend proximal to the second proximal face of the core, wherein the fourth bend is in a fourth direction opposite the third direction.

14. The medical device of claim 1, wherein a cross-sectional dimension of a proximal portion of the first channel is smaller than a cross-sectional dimension of a distal portion of the first channel, and a cross-sectional dimension of a proximal portion of the second channel is smaller than a cross-sectional dimension of a distal portion of the second channel.

15. A medical device comprising:
a distal tip that includes:
a light source;
an imaging device;
a first articulation member, wherein a distal end of the first articulation member is fixed to a first ferrule;
a second articulation member, wherein a distal end of the second articulation member is fixed to a second ferrule; and
a core defining a lumen extending along a longitudinal axis of the core, a first channel configured to receive a first articulation member, and a second channel configured to receive a second articulation member, wherein:
the first channel is parallel to the lumen and at least one of a first distal face or a first proximal face defined by the first channel is transverse to the lumen; and
the second channel is parallel to the lumen and at least one of a second distal face or a second proximal face defined by the second channel is transverse to the lumen.

16. The medical device of claim 15, wherein a proximal end of the first ferrule abuts the first distal face and a proximal end of the second ferrule abuts the second distal face.

17. The medical device of claim 15, further comprising:
a handle; and
an insertion portion extending distally from the handle;
wherein each of the first articulation member and the second articulation member extends from the handle through the insertion portion to the distal tip, and wherein the first articulation member includes a first bend distal to the first distal face, and the second articulation member includes a second bend distal to the second distal face.

18. A medical device comprising:
a handle;
an insertion portion extending distally from the handle to a distal tip and defining a first lumen, wherein a first articulation member and a second articulation member each extend from the handle through the insertion portion, wherein the distal tip includes:
a light source;
an imaging device; and
a core including a first channel that receives a distal portion of the first articulation member and a second channel that receives a distal portion of the second articulation member, wherein the first channel defines a first distal face and the second channel defines a second distal face;
wherein each of the light source and the imaging device is distal to the distal portion of each of the first articulation member and the second articulation member; and
wherein the first articulation member includes a first bend distal to the first distal face and the second articulation member includes a second bend distal to the second distal face.

19. The medical device of claim 18, wherein each of the first bend and the second bend is approximately 90 degrees to approximately 180 degrees.

20. The medical device of claim 18, wherein the core further includes:
a third channel adjacent to the first channel, wherein the third channel receives a portion of the first articulation member after the first bend; and
a fourth channel adjacent to the second channel, wherein the fourth channel receives a portion of the second articulation member after the first bend.
